(19) United States
(12) Patent Application Publication
(10) Pub. No.: US 2023/0373884 A1
(43) Pub. Date: Nov. 23, 2023

(54) METHOD AND SYSTEM FOR PRODUCING AROMATIC HYDROCARBONS

(71) Applicant: KOCH TECHNOLOGY SOLUTIONS, LLC, Wichita, KS (US)

(72) Inventors: Brant Lane Aggus, Corpus Christi, TX (US); Daniel Travis Shay, Newtown Square, PA (US); Tesia Valeska Chciuk, Wilmington, DE (US); Jinghuai Wu, Wilmington, DE (US)

(21) Appl. No.: 18/030,514

(22) PCT Filed: Oct. 1, 2021
(86) PCT No.: PCT/IB2021/059064

§ 371 Date: Apr. 6, 2023

Related U.S. Application Data
(60) Provisional application No. 63/107,176, filed on Oct. 29, 2020.

(51) Int. Cl. C07C 6/08

(52) U.S. Cl. CPC C07C 6/08

(57) ABSTRACT

Methods and systems for processing a first composition of a light hydrocarbon mixture with a 100° F. vapor pressure range from 2 to 51 psia into a second composition of aromatic hydrocarbons are provided. The method comprises contacting the first composition with a reaction zone comprising three or more operating fixed bed reactors in series, each reactor containing porous solid acid catalyst, said method further comprising the step of adding a toluene feedstock to said reaction zone under conditions in which the first composition is transformed to a second composition in an amount greater and at a percentage higher in aromatics than produced via a parallel or single reactor process with the same catalyst without the toluene feedstock, and/or contacting said second composition comprising heavy aromatics with said reaction zone to further increase amount and percentage of aromatics.

### FIELD

Methods and systems for processing a first composition comprising a light hydrocarbon mixture into a second composition of aromatic hydrocarbons are provided.

### BACKGROUND

Exploitation of shale gas and shale-oil resources in the United States, and elsewhere, involves production of substantial quantities of natural gas liquids (NGLs), sometimes referred to as condensate. Such condensate can comprise ethane, propane, butane, pentane and hexane, for example. While the methane content of shale gas can be used as a source of natural gas, maximizing the value of the “heavier” components is key to maintaining the profitability of shale gas production.

Condensate, in this context, is typically separated from natural gas, ethane and liquefied petroleum gas (or LPG) in gas separation plants. The condensate comprises straight-chain pentane and hexane and is both low octane and has a high vapor pressure. As such, it is generally unsuitable for use in the gasoline fuel pool. Instead, the condensate is used as a feedstock for olefin steam crackers as an alternative to ethane or refinery naphtha, accordingly commanding a significantly lower value than gasoline.

As refineries switch from heavier sources of crude oil to lighter crudes, which originate in shale deposits, the proportion of light paraffinic naphtha generally increases. As a result, the straight run naphtha produced from the crude fractionator contains large quantities of straight chain pentane and hexane, which has a relatively low octane number or unit and relatively high vapor pressure. Further processing of this stream to reduce sulfur content by hydrotreating further reduces the octane content as a result of hydrogenation of unsaturated species such as olefins.

One means by which it is possible to increase octane content is isomerization, which increases the proportion of branched paraffins. However, this has the effect of further increasing the vapor pressure of the stream and therefore is not a feasible route for use of the stream in gasoline blend stocks.

U.S. Pat. No. 3,960,978 discloses metalized (cation exchanged) zeolites, such as ZSM-5 & ZSM-11, that comprise metals such as Zn, Cr, Pt, Pd, Ni, and Re, for example, in a process technology referred to as M-Forming™ (Chen et al., 1986). The general understanding is that the ion exchange adds oligomerization capability to the aromatization functionality within the zeolite matrix and may enable the conversion of low molecular weight olefins, such as propylene, into oligomers and aromatics, via the catalyst's dehydrocyclization functionality. The U.S. refining industry has not however widely utilized this technology, presumably because it is not economically favorable and/or technically impractical or unduly complex.

Catalyst applications substantially involving crystalline zeolites are also known. For example, published U.S. patent application nos. 2010/0247391, 2010/0249474 and 2014/0024870 disclose processes for converting ethylene in a dilute ethylene stream to heavier hydrocarbons using amorphous silica alumina materials containing Group VIII & Group VIB metals in a fixed catalyst bed.

However, such catalyst beds are quickly deactivated by the deposition of coke.

Circulating fluidized bed reactors or moving bed reactors which allow for catalyst regeneration have been described. However, circulating fluidized beds or moving beds require engineering of an expensive solids transport system and the catalyst is subjected to mechanical forces stronger than in fixed beds.

EP3389842B1 discloses a process for converting liquefied petroleum gas of C2-C4 alkanes and essentially free of methane to higher hydrocarbons in a process of preferably 4-6 reaction zones of catalytic material operated in series at increasing temperature profiles, wherein not all reaction zones in the series are participating in the reaction.

US20170129827A1 discloses a process for converting natural gas with high methane content to higher hydrocarbon(s) including aromatic hydrocarbon(s) in 3 or more reaction zones of catalytic material operated in series, wherein not all reaction zones are participating in reaction.

WO2019/118825 discloses a method and system for processing light naptha into higher molecular weight paraffins, napthenics and aromatics using a multibed downflow reactor with a heterogeneous catalyst comprising pentasil zeolite.

CN105272803A and CN1234669C utilize toluene disproportionation with heavy aromatics (C9+A) to increase production of xylol and xylene.

CN1318359C discloses utilization of toluene disproportionation with heavy aromatics (C8+A and C9+A) and two zeolite catalysts to reduce indane content during production of hydrocarbons with 11 and more than 11 carbon atoms.

Zeolite catalysts for use in toluene disproportionation processes are also disclosed in U.S. Pat. Nos. 8,754,247 and 10,661,258.

Improved methods and systems are needed to efficiently produce more valuable hydrocarbon mixtures from C5 or greater hydrocarbon isomer streams.

### SUMMARY

An aspect of the present invention relates to a method for processing a first composition comprising a light hydrocarbon mixture to a second composition of aromatic hydrocarbons. In this method a first composition of a light hydrocarbon mixture with a 100° F. vapor pressure range from 2 to 51 psia is contacted with a reaction zone comprising three or more operating fixed bed reactors in series. Each reactor contains porous solid acid catalyst.

In one nonlimiting embodiment, the method further comprises adding a toluene feedstock to the reaction zone under conditions in which the first composition is transformed to a second composition in an amount greater and at a percentage higher in aromatics than produced via a parallel or single reactor process with the same catalyst without the toluene feedstock.

In another nonlimiting embodiment, the method further comprises contacting the second composition comprising heavy aromatics with the reaction zone to further increase amount and percentage of aromatics.

In yet another nonlimiting embodiment, the method further comprises both steps of adding a toluene feedstock to the reaction zone under conditions in which the first composition is transformed to the second composition in an amount greater and at a percentage higher in aromatics than produced via a parallel or single reactor process with the same catalyst without the toluene feedstock and contacting the second composition comprising heavy aromatics with the reaction zone to further increase amount and percentage of aromatics.

Another aspect of the present invention relates to a system for processing a first composition comprising a light hydrocarbon mixture to a second composition of aromatic hydrocarbons. The system comprises a reaction zone of three or more reactors in series with each reactor containing porous solid acid catalyst. In this system, each reactor is separately piped and valved so that a user can alternate the order in which the first composition is contacted with the three or more reactors. The system further comprises a means for providing uniform inlet temperatures across the reactors in series and a means for providing the first composition to the reactors.

In one nonlimiting embodiment, the system further comprises a means for adding a toluene feedstock to the reaction zone under conditions in which the first composition is transformed to the second composition in an amount greater and at a percentage higher in aromatics than produced via a parallel or single reactor process with the same catalyst without the toluene feedstock.

In another nonlimiting embodiment, the system further comprises a means for contacting the second composition comprising heavy aromatics with the reaction zone to further increase amount and percentage of aromatics.

In yet another nonlimiting embodiment, the system further comprises means for both adding a toluene feedstock to the reaction zone under conditions in which the first composition is transformed to the second composition in an amount greater and at a percentage higher in aromatics than produced via a parallel or single reactor process with the same catalyst without the toluene feedstock and means for contacting the second composition comprising heavy aromatics with the reaction zone to further increase amount and percentage of aromatics.

### BRIEF DESCRIPTION OF THE FIGURES

FIG. 1 is a diagram showing a typical parallel reactor implementation.

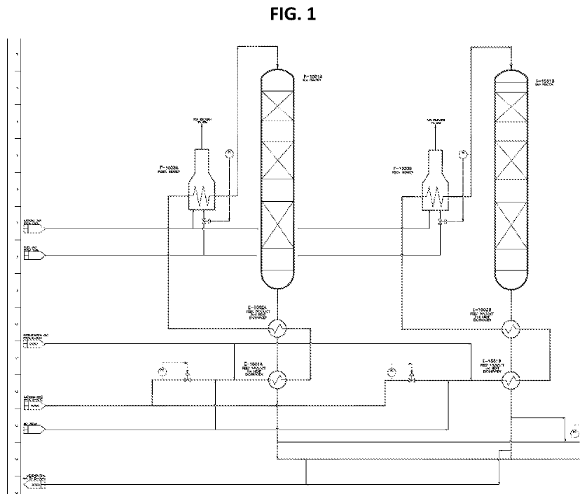

FIG. 2 is a diagram showing a nonlimiting embodiment of a system of the present invention with a series reactor configuration.

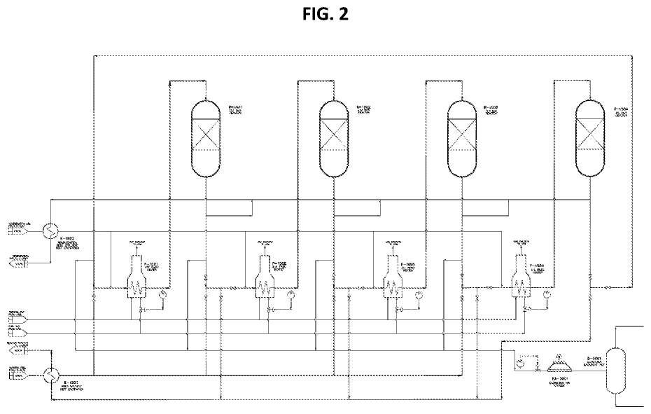

FIG. 3 is a line graph showing weight percent (wt. %) of aromatics in a reactor effluent with n-hexane feed.

### DETAILED DESCRIPTION

This disclosure relates to methods and systems for more efficient production of aromatic hydrocarbons from feed streams high in content of light hydrocarbon mixtures.

For purposes of the present invention, by “light hydrocarbon mixture” it is meant to include a hydrocarbon mixture with a 100° F. vapor pressure range from 2 to 51 psia.

Prior reactor systems for this process have been designed with parallel or single-train reactor design. The resulting implementation is that a reactor vessel may be taken out of service, and the hydrocarbon mixture described above directed to either the parallel train or the flow redirected away from the process entirely, while the out of service vessel is subjected to a sub-stoichiometric oxygen flow in a nitrogen diluent at high temperature, oxidizing carbonaceous deposit in the catalyst pores and subsequently removing it. To increase aromatization and octane number of the recovered hydrocarbon reactor product, operating temperature must be increased. The required frequency of the carbon burn process, which is required to maintain desired conversion of hydrocarbons to aromatics, increases as the operating temperature of the process is increased, to a point that frequency of regeneration will make the aforementioned parallel or single reactor train design uneconomic due to required catalyst quantity and reactor size.

In the method and systems of this disclosure, a first composition comprising a light hydrocarbon mixture is contacted with a reaction zone comprising three or more operating reactors in series under conditions in which the first composition is transformed to a second composition of aromatic hydrocarbons. In one nonlimiting embodiment, the three or more operating reactors are operating fixed bed reactors.

In one nonlimiting embodiment, the first composition is naptha derived from a crude oil distillation unit or from a natural gasoline or condensate.

In one nonlimiting embodiment, the second composition produced via the method and system of this disclosure comprises 15-55 wt % aromatic hydrocarbons.

In one nonlimiting embodiment, the second composition comprises benzene, xylene and toluene. In one nonlimiting embodiment, the second composition comprises higher percentages of benzene and p-xylene as compared to toluene.

Reactors in series of the reaction zone contain porous solid acid catalyst. Any porous solid acid catalyst used in parallel or single-train reactor systems for processing a feed stream of hydrocarbon isomers into aromatic hydrocarbons can be used in the methods and systems of this disclosure.

In one nonlimiting embodiment, the porous solid acid catalyst comprises silica, alumina, aluminosilicate or any mixture thereof.

In one nonlimiting embodiment, the catalyst comprises a synthetic zeolite or other mesoporous materials, such as, but not limited to MCM-41, which are widely used as catalysts in the petrochemical industry. Medium pore zeolites, such as, but not limited to, those from the ZSM-5 family, as well as Zeolite X, Y beta, ZSM-22 and ferrierite can be used. Nonlimiting examples of catalysts useful in the present invention include, but are not limited to those disclosed in U.S. Pat. Nos. 8,969,232; 9,192,925; 10,625,247; 10,272,420; and 10,611,645 and WO2019/118825, teachings of which are incorporated herein by reference in their entirety.

In addition to porosity, catalysts used in these methods and systems are selected based upon acidity, Si/Al ratio, types of catalytic active metals and location in the zeolite matrix.

Conditions in which the first composition of a light hydrocarbon mixtures is transformed to the second composition of aromatic hydrocarbons may be similar to those used for parallel or single-train reactor systems with the process operating in a vapor phase. Advantageously, however, in the method and system of this disclosure, the first composition can be transformed to the second composition at a temperature lower than the temperature required in a system with reactors of the same catalyst in parallel.

In one embodiment of this process, the inlet temperature for each operating reactor in series is maintained between 275° C. and 600° C.

In one nonlimiting embodiment, the reaction zone of each reactor in series is sized so that the outlet temperature remains above the minimum reaction temperature.

The method and system of this disclosure are surprisingly insensitive to pressure. Suitable ranges include 1 psig to 500 psig, for example, 25 to 100 psig.

In one nonlimiting embodiment of the method and system of this disclosure, contacting the first composition with the catalyst is performed in the presence of a toluene feedstock. Surprisingly, in the present of the toluene co-feed, aliphatics converted to aromatics with higher selectivity and yields as compared to similar processes without the toluene co-feed. Further, toluene disproportionation appears to be undiminished. Thus, without being limited to any specific theory or mechanism, the two reactions appear to co-exist without unduly interfering with each other. These results require severe temperature conditions in a highly endothermic process using catalysts that deactivate quickly at high temperature and cannot be performed in prior reactor systems for this process with parallel or single-train reactors.

In one nonlimiting embodiment, fresh toluene is used as the feedstock. In another nonlimiting embodiment, the toluene feedstock is recycled from the process.

In another nonlimiting embodiment of the method and system of this disclosure, the second composition is re-fed back into the reaction zone for further processing to increase aromatic content. In one nonlimiting embodiment, the second composition is re-fed back to one of the three or more reactors in series. In one nonlimiting embodiment, the second composition is fed to a separate reactor. In this nonlimiting embodiment, the catalyst used for the re-processing of the second composition will be a porous solid acid catalyst, which may be the same as or different to the catalyst used in the three or more reactors in series.

In yet another nonlimiting embodiment of the method and system of this disclosure, the first composition with the catalyst is performed in the presence of a toluene feedstock and the second composition is re-fed back into the reaction zone for further processing to increase aromatic content.

Optionally, the method and system may further comprise a hydrogen co-feed. In one nonlimiting embodiment, toluene is added in the presence of a molar excess added hydrogen to molar equivalent of toluene converted.

In one embodiment of the system of this disclosure, each of the three or more reactors in series containing porous solid acid catalyst of the reaction zone is separately piped and valved so that a user can alternate the order in which the first composition is contacted with the three or more reactors.

This system further comprises means for providing uniform inlet temperatures across the reactors in series. Nonlimiting examples of temperature control means include indirect heating via fired heater or heat exchange with another thermal medium.

This system further comprises means for providing the first composition to the three or more reactors in series. In one nonlimiting embodiment, the system further comprises a means for providing a toluene feedstock to these reactors and/or a means for re-feeding the second compositions to these reactors or another separate reactor in the reaction zone. Such means include, but are not limited to pumps, pipes and valving.

As shown herein, using this method and system, aromatic hydrocarbons are produced in the second composition in an amount greater and at a percentage higher in aromatics than produced via a parallel or single reactor process with the same catalyst without the toluene feedstock. Further, the method and system are characterized by high per-pass conversion and desirable selectivity to benzene and xylene.

Another advantage of this invention with the reactors in series is that less catalyst is required to produce a greater amount of aromatic hydrocarbons as compared to methods and systems with parallel or single-train reactor systems. In addition, there are lower costs associated with regeneration circuit equipment since smaller catalyst loads can be regenerated in less time.

Further, with this method and system, interstage reheating can be applied to enhance yield control and maximize catalyst activity and run length. Means for interstate heating which can be incorporated into the system include, but are not limited to indirect heating via electric or fired furnace, or cross-exchange with a fluidized heating medium.

A further advantage of the reactors in series is that the sequence of contacting the first composition with the three or more reactors is variable. In one nonlimiting embodiment of this method and system, the first composition is first contacted with the reactor with least active catalyst in the series.

An additional advantage of this method and system is that one reactor in the series may be out of service for catalyst regeneration or replacement without affecting production. The ability to continuously regenerate catalyst without reduced unit throughput or shutdown also provides for increased operating temperature and higher severity is desired.

Further, operating pressure for the methods and systems of this disclosure range from 30 psig to 300 psig or 100 psig to 250 psig.

In addition, implementing a manifold of smaller reactors operated in series, rather than in parallel, creates an arrangement where a shorter run length can be tolerated for lower cost.

Further, importantly, these methods and systems tolerate high severity operation through their utilization of multiple reactor vessels allowing for interstage heating to be applied, keeping the endothermic net process above the minimum temperature required to initiate aromatization. In addition, a lower inlet temperature can be applied, as the conversion of paraffins to aromatics is limited in each reactor.

Hence, using methods and system of this disclosure, higher product octane or aromatic content is achievable, with smaller and lower capital cost regeneration equipment required.

The following nonlimiting examples are provided to further illustrate the present invention.

### EXAMPLES

10 grams of 16 mesh zeolite catalyst loaded in a 0.500 inch×0.0049 inch wall 316SS tube was inserted in an isothermal heating block set to 450 C while flowing 5 liters/hour nitrogen at 25 psig maintained by a diaphragm style back pressure regulator located at the reactor exhaust. Once catalyst temperature reached equilibrium, nitrogen flow was discontinued and hexane was introduced via a syringe pump at 2.5 grams/hr. 100% of the product exited the reactor via electrically heated lines maintaining a single gas phase and was reduced to atmospheric pressure and analyze on-line by an Agilent 7890B GC equipped with a 100 meter DHA boiling point column. The reactor product composition after 50 hours on steam is presented in the table below.

### Final Reactor Product PIONA (Carbon Number vs Normal Paraffin, Iso Paraffin, Olefin, Naphthalene, Aromatics)

PONA TABLE

| wt % | P | I | O | N | A |
|---|---|---|---|---|---|
| C1 | 10.30 | 0 | 0 | 0 | 0 |
| C2 | 15.32 | 0 | 0.46 | 0 | 0 |
| C3 | 17.46 | 0 | 0 | 0 | 0 |
| C4 | 0.45 | 0.29 | 0 | 0 | 0 |
| C5 | 0 | 0 | 0 | 0 | 0 |
| C6 | 0 | 0 | 0 | 0 | 14.10 |
| C7 | 0 | 0 | 0 | 0 | 21.42 |
| C8 | 0 | 0 | 0 | 0 | 9.51 |
| C9 | 0 | 0 | 0 | 0 | 1.46 |
| C10 | 0 | 0 | 0 | 0 | 3.38 |
| C11 | 0 | 0 | 0 | 0 | 1.09 |
| C12 | 0 | 0 | 0 | 0 | 4.77 |

Methodology: The laboratory yields were scaled up via Aspen HYSYS simulation to design a theoretical 2,500 bpsd facility, with the reactor operating temperatures obtained via Gibbs energy equilibrium of reactor feed and products.

### Example 1

A system with 5 reactors in series. 4 online during normal operation, 1 offline for regeneration or standby. Regeneration cycle of 7 days. Reactor switch every 15 days.

| | Reactor #1 | | | | | |
|---|---|---|---|---|---|---|
| | (Lead Reactor) | Reactor #2 | Reactor #3 | Reactor #4 | Reactor #5 | Total |
| Required | 770 | 824 | 878 | 932 | Offline - In | |
| reactor Inlet | | | | | Regeneration | |
| Temperature | | | | | | |
| C5+ Yield (wt % | 11.1 | 22.3 | 39.0 | 55.7 | | |
| of feed) | | | | | | |
| C5+ Product | 100 | 100 | 100 | 100 | | |
| Aromatic | | | | | | |
| Content (wt %) | | | | | | |
| Reactor | 213 | 174 | 248 | 143 | | |
| Endotherm (F.) | | | | | | |
| Charge heater duty | 23.6 | 16.4 | 12.7 | 12.7 | | 65.4 |
| (MMBTU/hr) | | | | | | |
| Catalyst Load, | 12000 | 12000 | 12000 | 12000 | 12000 | 64,000 |
| each Reactor (lb) | | | | | | |

### Comparative Example #1

A system with 2 reactors in parallel, both online during normal operation, 1 taken offline every 45 days for regeneration was used. Both reactors were designed for 100% feed flow and design conversion. Reactor switch every 45 days.

| | |
|---|---|
| C5+ Yield (wt % of feed) | 55.7 |
| Required Reactor Inlet Temperature (F.)* | 1174 |
| Reactor Endotherm (F.)** | 639 |
| Feed Aromatic Content (wt %) | 0 |
| C5+ Product Aromatic Content (wt %) | 100 |
| Charge heater duty (MMBTU/hr) | 65.1 |
| Catalyst load (each reactor) | 48,000 lb |
| Catalyst load (total) | 96,000 lb |

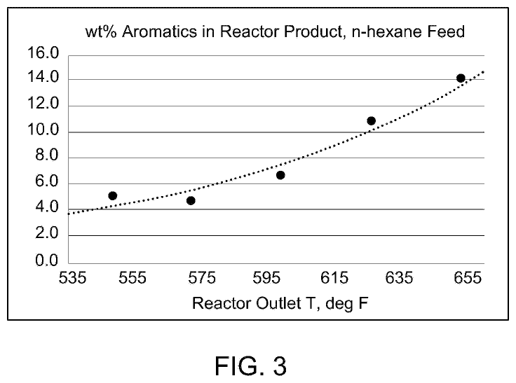

*= This is theoretical. Approximately 1022° F. is the maximum temperature allowable before thermal cracking becomes predominant. The formation of coke in zeolite-catalyzed aromatization processes at high temperature is well known. See for example, U.S. Pat. No. 5,866,744.
**= Based on Gibbs free energy. 726° F. inlet temperature is required to maintain minimum required temperature for aromatization. Laboratory analysis has found the minimum operating temperature to maintain aromatization to be approximately 535° F. at 0.5 psig reactor operating pressure (see FIG. 3).

### Example 2: Toluene Disproportionation

5 grams of 16 mesh zeolite catalyst loaded in a 0.500 inch×0.0049 inch wall 316SS tube was inserted in an isothermal heating block set to 450 C while flowing 5 liters/hour nitrogen at 25 psig maintained by a diaphragm style back pressure regulator located at the reactor exhaust. Once catalyst temperature reached equilibrium, nitrogen flow was reduced to 1.7 liters/hour and toluene introduced via a syringe pump at 0.42 grams/hr. 100% of the product exited the reactor via electrically heated lines maintaining a single gas phase and was reduced to atmospheric pressure and analyze on-line by an Agilent 7890B GC equipped with a 100 meter DHA boiling point column. The reactor product composition after 50 hours on steam is presented in the table below.

| | |
|---|---|
| Benzene (Total wt %) | 4.83 |
| Toluene (Total wt %) | 84.97 |
| Toluene Conversion (wt %) | 15.03 |
| Xylenes (Total wt %) | 6.18 |
| Total Disproportionation Products (Total wt %) | 11.00 |
| Total Nonselective Products (Total wt %) | 4.03 |
| Total Aromatic C9+ Products(Total wt %) | 2.86 |
| Total Paraffins (Total wt %) | 1.08 |
| Total Olefins (Total wt %) | 0.08 |

1. A method for processing a first composition comprising a light hydrocarbon mixture with a 100° F. vapor pressure range from 2 to 51 psia, said method comprising contacting the first composition with a reaction zone comprising three or more operating fixed bed reactors in series, each reactor containing porous solid acid catalyst, said method further comprising the step of:
(i) adding a toluene feedstock to said reaction zone under conditions in which the first composition is transformed to a second composition in an amount greater and at a percentage higher in aromatics than produced via a parallel or single reactor process with the same catalyst without the toluene feedstock, and/or
(ii) contacting said second composition comprising heavy aromatics with said reaction zone to further increase amount and percentage of aromatics.

2. The method of claim 1, wherein said toluene feedstock is added in the presence of zero or a molar excess added hydrogen to molar equivalent of toluene converted.

3. The method according to claim 1, wherein said second composition comprising heavy aromatics is contacted with a reactor in said reaction zone which is separate from said three or more operating reactors in series in said reaction zone.

4. The method according to claim 3, wherein said separate reactor in said reaction zone contains a porous solid acid catalyst.

5. The method of claim 1, wherein the first composition is transformed to the second composition with an inlet temperature lower than the temperature required in a system with reactors of the same catalyst in parallel.

6. The method of claim 1 wherein the inlet temperature for each operating reactor in series is maintained between 275° C. and 600° C.

7. The method of claim 1, wherein less porous solid acid catalyst is required to produce a greater amount of aromatics as compared to a system with reactors of the same catalyst in parallel.

8. The method of claim 1, wherein the first composition is naptha derived from a crude oil distillation unit or from a natural gasoline or condensate.

9. The method of claim 1, wherein sequence of contacting the first composition with the three or more reactors is variable.

10. The method of claim 1 wherein the first composition is first contacted with the reactor with least active catalyst in the series.

11. The method of claim 1, wherein at least one reactor is out of service for catalyst regeneration or replacement.

12. The method of claim 1 wherein the second composition comprises 15-55 wt % aromatic hydrocarbons.

13. The method of claim 1 wherein the second composition comprises benzene, xylene and toluene.

14. The method of claim 1 further comprising recycling of the toluene feedstock.

15. The method of claim 1, wherein said toluene is distilled from said second composition comprising benzene, xylene and toluene.

16. (canceled)

17. A system for processing a first composition comprising a light hydrocarbon mixture with a 100° F. vapor pressure range from 2 to 51 psia to a second composition of aromatic hydrocarbons, said system comprising a reaction zone comprising three or more operating fixed bed reactors in series, wherein each reactor contains porous solid acid catalyst and is separately piped and valved so that a user can alternate order in which the first composition is contacted with the three or more reactors, a means for providing uniform inlet temperatures across the reactors in series, a means for providing the first composition to the reactors and a means for providing toluene feedstock to the reactors and/or a means for contacting said second composition comprising heavy aromatics with said reaction zone to further increase amount and percentage of aromatics, wherein said system transforms the first composition to a second composition in an amount greater and at a percentage higher in aromatics than produced via a system of parallel reactors with the same catalyst without the toluene feedstock.

18. The system of claim 17, wherein said toluene feedstock is added in the presence of zero or a molar excess added hydrogen to molar equivalent of toluene converted.

19. The system of claim 17 comprising a reactor which is separate from said three or more operating reactors in series in said reaction zone for contacting said second composition comprising heavy aromatics to further increase amount and percentage of aromatics.

20. (canceled)

21. The system of claim 17, wherein the first composition is transformed to the second composition at a temperature lower than the temperature required in a system with reactors of the same catalyst in parallel.

22. The system of claim 17 comprising less catalyst to produce a similar amount of aromatics as compared to a system with reactors of the same catalyst in parallel without a toluene feedstock.

23-24. (canceled)
